(19) United States
(12) Patent Application Publication
(10) Pub. No.: US 2026/0053528 A1
(43) Pub. Date: Feb. 26, 2026

(54) Imaging-Guided Needle Guiding Device with Real-Time Attitude Monitoring, Feedback System, and Precision Calibration Method

(71) Applicants: Run Zhou Ye, Rochester, MN (US); En Zhou Ye, Montreal (CA); En Hui Ye, Montreal (CA); Joseph Ye, Montreal (CA); Jacob Ye, Montreal (CA); Bin Zhang, Montreal (CA); Ming Ye, Montreal (CA)

(72) Inventors: Run Zhou Ye, Rochester, MN (US); En Zhou Ye, Montreal (CA); En Hui Ye, Montreal (CA); Joseph Ye, Montreal (CA); Jacob Ye, Montreal (CA); Bin Zhang, Montreal (CA); Ming Ye, Montreal (CA)

(21) Appl. No.: 18/814,558

(22) Filed: Aug. 25, 2024

(51) Int. Cl. A61B 17/34; A61B 17/00; A61B 34/10; A61B 34/20

(52) U.S. Cl. CPC A61B 17/3403; A61B 34/20; A61B 2017/00221; A61B 2017/0023; A61B 2017/00725; A61B 2034/107; A61B 2034/2048; A61B 2034/2065; A61B 2034/2072

(57) ABSTRACT

A needle guiding device for imaging-guided procedures features sensors to monitor the needle's attitude and a calibration tool fixed to the imaging machine for alignment. The device offers customizable feedback, including visual, audible, and haptic alerts, to maintain the planned insertion trajectory. The calibration tool, which may include reference surfaces that are perpendicular, non-perpendicular, or feature a groove, ensures accurate alignment. The device can log procedural data and is compatible with both single-use and reusable configurations. It is designed for use with one needle at a time, with options to use multiple devices for multiple needles or sequentially against different needles.

### FIELD OF THE INVENTION

This invention relates to advanced medical devices and systems, particularly to a device that enhances the precision of needle insertion procedures in human or animal subjects. The device provides real-time monitoring of the needle's orientation (attitude), offers continuous feedback to the practitioner, and includes a detailed calibration method to align the device with the imaging system, ensuring accuracy throughout the procedure. The device is designed to be used with one needle at a time, either through a clamping mechanism (107) or a cylindrical design with a long groove (203). If the procedure involves multiple needles, the user can either use multiple guiding devices (101), each dedicated to a single needle (102), or place the device sequentially against different needles.

### BACKGROUND OF THE INVENTION

Accurate needle placement is essential in a wide range of medical procedures, including biopsies, drug delivery, catheterization, cryoablation, and various forms of minimally invasive surgery. The success of these procedures heavily depends on the precise alignment and insertion of the needle (102) according to pre-planned trajectories based on imaging data. Traditional methods often rely on the skill and experience of the practitioner, which can lead to variability in outcomes, particularly in complex or high-risk procedures.

While some existing technologies provide guidance, they often lack a standardized calibration method that ensures the alignment of the needle's orientation with the imaging system used for planning. This gap can lead to misalignment and inaccuracies during needle insertion, increasing the risk of procedural errors. The invention described here addresses these challenges by providing a needle guiding device (101) that integrates real-time monitoring, feedback mechanisms, and a calibration process, making it a comprehensive solution for improving accuracy in needle-based medical procedures. The device is designed for single-needle use, with options for either a clamping mechanism (107) or a cylindrical design with a long groove (203). If the procedure involves multiple needles, the user can either use multiple guiding devices (101), each dedicated to a different needle, or place the device sequentially against different needles.

### SUMMARY OF THE INVENTION

This invention offers a device (101) that attaches to a single needle (102) at a time, monitors its attitude in real-time, and provides continuous feedback to the user to maintain alignment with pre-planned insertion angles. The device is equipped with multiple sensors, including an accelerometer (104), gyroscope (105), and magnetometer (106). The device can operate in different modes: in one mode, the pre-planned angles are sent to the device, and the device itself compares these with the real-time orientation of the needle (102), providing feedback without transmitting data externally. In another mode, if real-time image overlay is desired, sensor data can be transmitted to an external processor, and the actual real-time angles can be displayed on an external display, such as a computer or smartphone.

A key feature of the invention is the calibration method that aligns the device (101) with the imaging machine's (302) coordinate system. The calibration tool (301) is designed to be attached to the imaging machine (302) or machine table (307). It features two rigid reference surfaces (303, 304) that may be perpendicular, non-perpendicular, or feature a groove (305) to fit the device (101). The tool can be permanently attached using an adhesive layer (305) or secured with screws (306) or similar fasteners. The needle guiding device (101) is then placed on this tool, allowing the sensors to calibrate to the imaging system's reference angles. The device is designed to be used with one needle (102) at a time, either through a clamping mechanism (107) or a cylindrical design with a long groove (203). If the procedure involves multiple needles, the user can either use multiple guiding devices (101), each dedicated to a single needle (102), or place the device sequentially against different needles.

### BRIEF DESCRIPTION OF THE DRAWINGS

FIG. 1: Illustration of the needle guiding device (101) attached to a single needle (102). The figure shows the housing of the device, which encloses the integrated sensors (103) including an accelerometer (104), gyroscope (105), and magnetometer (106). The device's attachment mechanism (107) is depicted clamping onto the needle, ensuring stability along the needle's axis. The figure also shows the edge or tip (108) of the clamp that can be positioned against the needle for angle measurement without full attachment.

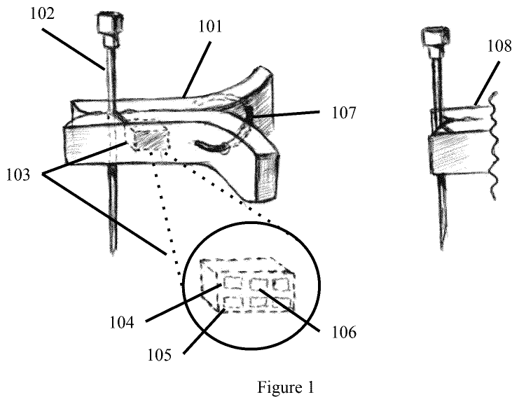

FIG. 2: Detailed view of the alternative cylindrical design of the needle guiding device (201) with the integrated sensor module (202), featuring a long groove (203) that is placed against a single needle (204) without clamping. The figure shows how the device is positioned to fit snugly against the needle, providing a stable reference point for orientation monitoring.

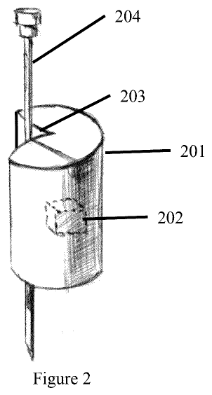

FIG. 3: Detailed view of the calibration tool (301), which can be permanently fixed to either the imaging machine (302) or the machine table (307). The tool can be secured using an adhesive layer (305) or mechanical fasteners such as screws (306). The calibration tool includes two reference surfaces (303, 304), which may be perpendicular, non-perpendicular, or feature a groove (305) designed to fit the needle guiding device (101) for precise alignment.

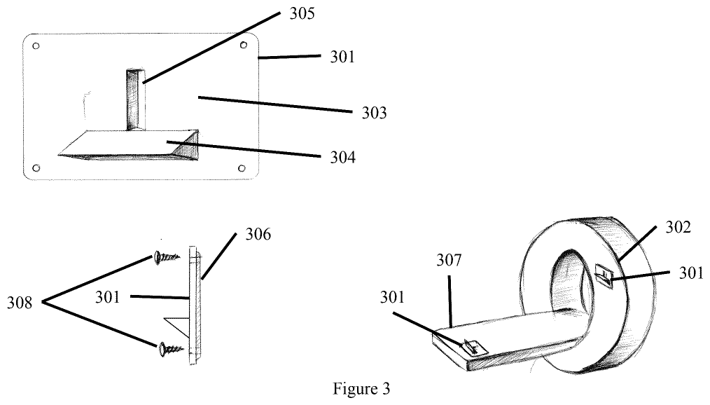

FIG. 4: Schematic diagram of the feedback system integrated into the needle guiding device (101). The diagram illustrates the visual indicators (401), such as LEDs, which provide real-time feedback on the needle's alignment. Audible alerts (402) are represented by a speaker symbol, while haptic feedback is shown as a vibration module within the device or a connected wearable (403).

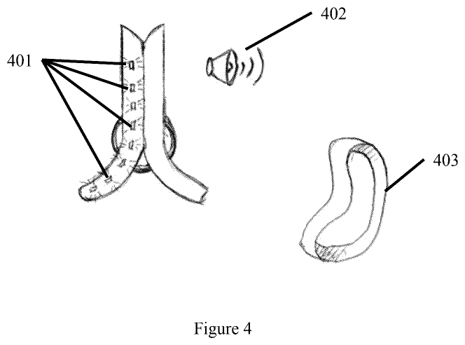

FIG. 5: Flowchart of the operational methodology for the device. The process begins with pre-insertion planning (501), where the user determines the needle's insertion path using imaging data. The needle guiding device (101) is then calibrated (502) using the calibration tool (301) before being attached to the needle (503). Real-time monitoring (504) and feedback (505) occur during the procedure, with the device (101) continuously comparing the needle's orientation to the planned trajectory, sending data to an external processor (506) to display the real-time angles on an external display.

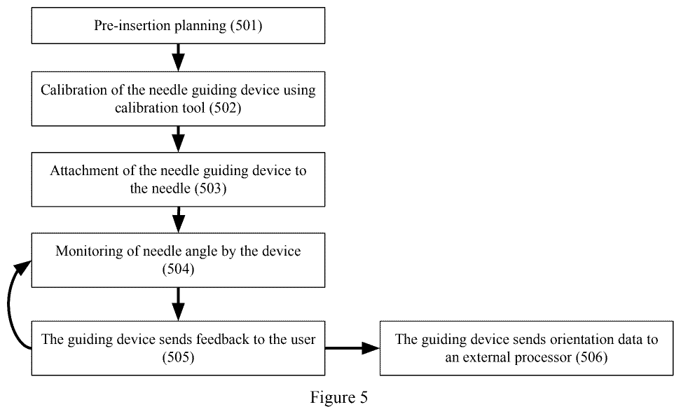

### DETAILED DESCRIPTION OF THE INVENTION

1. Device Construction: The needle guiding device (101) is designed with the following key components and features:

### Needle Attachment Mechanism:

In one embodiment, the device (101) is designed to be securely attached to a single needle (102) using a clamp mechanism (107). This mechanism ensures that the device remains stable along the needle's axis.

In another embodiment, the device (201) may feature a cylindrical design with a long groove (203) that allows the user to place it against a single needle (204) without clamping. This groove (203) is designed to fit against the needle, providing a stable reference point for angle measurement and orientation monitoring.

The device (101) is designed for single-needle use only. If the procedure involves multiple needles, the user can either use multiple guiding devices (101), each dedicated to one needle (102), or place the device sequentially against different needles.

### Sensor Integration:

Accelerometer (104): The accelerometer measures the needle's (102) static and dynamic orientation relative to gravity. This sensor provides data on the tilt and inclination of the needle as it is inserted into the tissue.

Gyroscope (105): The gyroscope tracks the needle's (102) angular velocity, allowing the device (101) to detect any rotational movements. This is important for maintaining the correct needle orientation during complex insertions that require precise angular control.

Magnetometer (106): The magnetometer provides orientation data by detecting the needle's (102) position relative to the Earth's magnetic field. This data complements the accelerometer (104) and gyroscope (105) readings, helping to correct for any drift or inaccuracies in the other sensors.

Temperature and Pressure Sensors: Additional sensors can be integrated into the device (101) to monitor environmental conditions such as temperature and pressure.

### Electronics and Power Supply:

The device (101) includes a microcontroller that processes the sensor data in real-time. The microcontroller is programmed with algorithms that filter and fuse the data from the various sensors, providing accurate and reliable readings of the needle's orientation.

Depending on the mode of operation, the device (101) can either transmit the processed data to an external processor (506) or handle all comparisons and feedback internally. If the real-time image overlay is not required, the device can operate independently, using pre-loaded angles to provide feedback.

The device (101) is powered by a compact, high-capacity battery designed to last for the duration of standard medical procedures. Optionally, the device can include a wired power option for procedures where uninterrupted power is critical.

### Device Durability and Usability:

The device (101) can be designed as either a single-use or reusable instrument. For single use, the device is made from materials that ensure sterility and reliability for one procedure. For reusable applications, the device is constructed from durable, autoclavable materials that withstand multiple sterilization cycles without compromising the integrity of the sensors or electronics.

2. Calibration Method: To ensure the device's (101) accuracy during needle insertion, a calibration method is included:

### Calibration Tool Design:

The calibration tool (301) is a precision-engineered device featuring two rigid reference surfaces (303, 304) that may be perpendicular, non-perpendicular, or feature a groove (305) for fitting the device (101). The tool can be permanently attached to the imaging machine (302) or machine table (307) using an adhesive layer (305) or secured with screws (306) or similar fasteners. These surfaces are fabricated to ensure accurate alignment with the imaging machine's (302) coordinate system. Once attached, the calibration tool (301) remains fixed, providing consistent reference angles for future procedures.

### Calibration Process:

Permanent Placement of the Calibration Tool: The calibration tool (301) is attached onto a flat, stable surface of the imaging machine (302) or machine table (307), securing it with either a strong adhesive or mechanical fasteners such as screws (306). Once attached, the calibration tool (301) remains permanently fixed to provide consistent reference planes.

Device Calibration: Before each procedure, the needle guiding device (101) is first calibrated using the method described above. The device (101) is placed on the calibration tool (301), and the sensors measure the angles of the reference surfaces (303, 304), establishing baseline orientation data. The microcontroller processes this data to calibrate the sensors, ensuring they are aligned with the imaging machine's (302) reference angles. This calibration step is critical for ensuring that the real-time data provided during the procedure accurately reflects the needle's (102) true position relative to the imaging data.

Verification: The external processor (506) (e.g., a smartphone, computer, or other external display) guides the user through the calibration process. Once the calibration is verified, the system signals that the device (101) is ready for use, either through visual cues on the display or audible notifications.

3. Operational Methodology: The operational methodology of the device (101) involves the following steps:

### Pre-Insertion Planning:

The procedure begins with the user planning the needle's insertion path using detailed imaging data, such as MRI, CT, or X-ray imaging. These images are used to determine the optimal angles and depth for the needle's (102) insertion, taking into account the anatomy and any specific procedural requirements.

The planned angles are recorded and stored in the external processor (506) or directly uploaded to the needle guiding device (101), which will later use this data to compare against the real-time orientation of the needle (102) during insertion.

### Device Calibration and Attachment:

After planning, the device (101) is first calibrated using the method described above, ensuring that the sensor readings are aligned with the imaging machine's (302) coordinate system. This calibration ensures that the data provided by the device (101) during the procedure will be accurate, reflecting the true orientation of the needle (102) relative to the planned trajectory.

The user then attaches the device (101) to the needle (102) using the secure attachment mechanism (107), or, in the case of the cylindrical design, places the device (201) against the needle (204) using the long groove (203). Alternatively, the user may place the tip or edge of the clamp (108) against the needle without fully clamping it if the user needs to verify the angle of the needle at any time during the procedure. The user can recalibrate the needle guiding device (101) at any time during the procedure.

The device (101) is designed for single-needle use only. If the procedure involves multiple needles, the user can either use multiple guiding devices (101), each dedicated to a different needle, or place the device sequentially against different needles.

### Real-Time Monitoring and Feedback:

During the needle insertion, the device (101) continuously monitors the needle's (102) attitude, using the calibrated sensors to provide real-time data on its orientation and movement.

The external processor or the device itself compares the real-time data to the pre-planned angles stored during the planning phase. The actual real-time angles can be displayed on an external display, such as a computer or smartphone, allowing the practitioner to continuously monitor the needle's (102) orientation.

If the needle deviates from the planned trajectory, the device (101) provides immediate feedback through multiple channels:
Visual Indicators: A series of light-emitting diodes (LEDs) on the device (101) or visual indicators on the external processor's display can provide continuous feedback on the needle's (102) alignment. These indicators change color or blink to alert the user to any deviation from the planned path.
Audible Alerts: The device (101) emits beeps or tones that increase in frequency or intensity as the needle deviates from the planned trajectory. This auditory feedback helps the practitioner make quick adjustments without needing to take their eyes off the patient or imaging screen.
Haptic Feedback: Vibrations in the device (101) or in a connected wearable (403), such as a wristband, alert the user to misalignment.

The feedback system is fully customizable, allowing the user to adjust the sensitivity of the alerts and the types of feedback provided based on their preferences and the specific requirements of the procedure.

### Post-Procedure Analysis:

After the procedure, the data related to the needle's movement and alignment can be reviewed in detail. This allows the practitioner to analyze the procedure's accuracy and refine their technique for future insertions.

1. A needle guiding device for use in imaging-guided procedures, comprising:
A housing attachable to or positionable against a single needle at a time;
At least one sensor configured to measure the orientation and movement of the needle;
A communication module for optionally transmitting sensor data to an external processor;
A feedback system providing real-time guidance to the user during needle insertion;
A calibration tool with at least two reference surfaces or may include a groove to fit the device, and is fixed to the imaging machine to align the needle guiding device with the imaging machine's coordinate system.

2. The device of claim 1, wherein the sensor includes any combination of accelerometer, gyroscope, magnetometer, and optionally additional sensors for temperature and pressure monitoring.

3. The device of claim 1, wherein the needle attachment mechanism allows the device to clamp onto a single needle or, in an alternative design, the device comprises a cylindrical shape with a long groove that is placed against the needle without clamping.

4. The device of claim 1, wherein the feedback system comprises any combination of visual indicators, audible alerts, and haptic feedback that guide the user to maintain the needle's alignment with the pre-planned trajectory.

5. The device of claim 1, further comprising a wireless communication module configured for Bluetooth or Wi-Fi connectivity to an external processor, with an optional wired communication mode.

6. The device of claim 1, wherein the external processor includes software for guiding the calibration process, monitoring real-time needle data, and confirming correct alignment with the imaging machine's coordinate system.

7. The device of claim 1, wherein the calibration process includes aligning the device with the reference surfaces of the calibration tool, which may be perpendicular, non-perpendicular, or feature a groove for fitting the device, to ensure accurate sensor calibration, with the calibration tool being fixed to the imaging machine.

8. The device of claim 1, wherein the device logs all procedural data for post-procedure analysis and training.

9. The device of claim 1, wherein the device integrates with imaging systems to provide real-time visual overlays of the needle's path relative to the planned trajectory on the imaging screen.

10. The device of claim 1, wherein the feedback system is programmable, allowing users to set specific thresholds for visual, audible, and haptic feedback based on the procedure's requirements.

11. The device of claim 1, further comprising a modular design that allows for easy replacement or upgrade of sensors and electronic components without requiring specialized tools.

12. The device of claim 1, wherein the calibration tool is fixed to the imaging machine and is portable across different imaging machines, ensuring the device can be calibrated in various clinical environments.

13. The device of claim 1, wherein the device is designed for single-use, made from materials that ensure sterility and reliability for one procedure.

14. The device of claim 1, wherein the device is designed for reusable applications, made from durable, autoclavable materials capable of withstanding multiple sterilization cycles without compromising the integrity of the sensors or electronics.

15. The device of claim 1, wherein the device is designed for use with one needle at a time, with the option to either use multiple guiding devices, each dedicated to a different needle during procedures involving multiple needles, or to place the device sequentially against different needles.

16. The device of claim 1, wherein the device is capable of displaying actual real-time angles on an external display, such as a computer or smartphone.
